(19) United States
(12) Patent Application Publication
(10) Pub. No.: US 2023/0389829 A1
(43) Pub. Date: Dec. 7, 2023

(54) WAKE-UP CIRCUIT

(71) Applicant: AZOTEQ HOLDINGS LIMITED, Nicosia (CY)

(72) Inventors: Frederick Johannes BRUWER, Paarl (ZA); Hein Barend VAN HEERDEN, Paarl (ZA); Douw Gerbrandt VAN DER MERWE, Paarl (ZA); Jacobus Daniel VAN WYK, Paarl (ZA)

(21) Appl. No.: 18/204,990

(22) Filed: Jun. 2, 2023

(30) Foreign Application Priority Data
Jun. 3, 2022 (ZA) 2022/06184

(51) Int. Cl. A61B 5/145; A61B 5/1455

(52) U.S. Cl. CPC A61B 5/14532; A61B 5/1455; A61B 2560/0209

(57) ABSTRACT

A wake-up circuit which includes a battery, a switch, an additional circuit and a detector which, responsive to incident light above a threshold level, closes the switch thereby to connect the battery to the additional circuit.

### CROSS-REFERENCE TO RELATED APPLICATION

The present application claims priority from South Africa application ZA 2022/06184, filed Jun. 3, 2022, contents of which is hereby incorporated by reference into this application.

### BACKGROUND OF THE INVENTION

Some electronic products have the requirement that batteries should already be inserted and connected during manufacturing. Naturally, this may cause battery depletion. If such products are stored for extended periods before sale, their batteries may discharge to unacceptable levels, or even run down completely.

For example, blood glucose monitoring units are often stored in sachets or in one or other form of sealed packaging. When a user removes the monitoring unit from its sachet the battery should have sufficient charge to allow use for the intended monitoring period. Users may expect the unit to detect when it is removed and applied, without the requirement for battery insertion. Some glucose monitoring units are in a sleep-mode when packaged, and require a wake-up action when they are removed from their packaging and applied. The sleep-mode should consume small enough amounts of power to ensure the battery maintains sufficient charge to allow the intended operating period even at the end of the shelf life of the monitoring unit.

Some prior art blood glucose monitoring units use Near Field Communication (NFC) technology to wake the unit up after removal from its packaging. For example, a user may utilize his or her smartphone to connect via NFC to the monitoring unit for wake-up. However, NFC transceivers and circuitry may introduce additional cost, and may require a fair amount of printed circuit board (PCB) real estate for the NFC coil. In addition, some users may not have access to NFC enabled smartphones or devices, thereby excluding them from the use of such blood glucose monitoring units.

A need exists for a wake-up circuit which consumes extremely low amounts of battery power, requires less PCB area and which is highly cost-effective. The present invention may address some or all of these needs.

### SUMMARY OF THE INVENTION

In an effort to clarify the disclosure of the present invention, the following summary is presented. This should not be construed as limiting to the claims of the invention as it is merely used to support clarity of disclosure. A large number of alternative embodiments may exist that fall within the spirit and scope of the present invention, as may be recognised by one skilled in the relevant arts. This summary is not intended to identify key or critical elements of the disclosed subject matter, nor is it intended to delineate the scope of the present invention or its the claims. It is intended to present a number of concepts in a simplified form to assist with the overall disclosure of the present invention

Herein, “or” is used to convey inclusive and not exclusive, unless expressly indicated otherwise or indicated otherwise by context. Therefore, herein, “A or B” may mean “A, B, or both,” unless expressly indicated otherwise or indicated otherwise by context. In addition, “and” is used to convey both joint and several, unless expressly indicated otherwise or indicated otherwise by context. Therefore, “A and B” may mean “A and B, jointly or severally,” unless expressly indicated otherwise or indicated otherwise by context

In a first exemplary embodiment of the present invention, a wake-up circuit may be connected to a positive and negative terminal of a battery, wherein the wake-up circuit may have an output terminal or terminals for the control of a power switch or switches, or for control of other electronic circuits, for example by transmitting the one or other signal to an input of said other circuit, and may further comprise a detector and a detection circuit sensitive to electromagnetic radiation, for example sensitive to light incident on said detector. The power switch may be used to selectively allow power to be transferred from said battery to the one or other load, for example to an electronic circuit of a product. Said wake-up circuit may control the power switch, or another electronic circuit, dependent on an amount of electromagnetic radiation received by said detector. For example, when the detector or a part of it receives a first amount of light which is less than a threshold, the wake-up circuit may control said power switch, or another circuit, via said output terminal to cause the power switch to be in an open state, or a substantially open state, thus preventing transfer of power from the battery to said load. Alternatively, when said detector receives the first amount of light which is less than said threshold, the wake-up circuit may provide the one or other signal to said another electronic circuit which may result in said circuit entering or maintaining a sleep or low-power mode. When said detector receives a second amount of light which is more than the threshold, the wake-up circuit may control said power switch, or another circuit, via said output terminal to cause the power switch to be in a closed state, or a substantially closed state, thereby allowing power to be transferred from said battery to said load for consumption by the load, or another circuit. Alternatively, when said detector receives said second amount of light which is more than the threshold, the wake-up circuit may provide the one or other signal to said another electronic circuit which may result in the circuit entering or maintaining a higher power consumption mode.

The load may also control said power switch in conjunction with, or apart from the wake-up circuit to allow or block transfer of electrical power from the battery, or another source of power, to said load. Alternatively, said another electronic circuit may control the state of said wake-up circuit in the one or other manner.

A related second exemplary embodiment may comprise a battery powered body parameter monitoring unit, for example a blood glucose monitoring unit, which may utilize an optical wake-up circuit to detect removal from its packaging, and to subsequently wake the unit, or activate it. For example, to subsequently connect monitoring and communication circuitry of the unit to said battery, or to a power source dependent on said battery. The monitoring unit may be for adhering to a user's body, with a small pin inserted into the skin of the user for measuring blood glucose levels, as is known in the art. A case or housing of the monitoring unit may comprise a window, aperture or opening, which may be sealed against ingress of liquids, moisture or other matter, said window allowing light to reach a light sensitive detector within the monitoring unit. Said window, aperture or opening may be blocked or masked when the monitoring unit is packaged, thus preventing, or substantially preventing, light from reaching said detector. When the glucose monitoring unit is removed from its packaging, ambient or other light may reach said detector. This may cause circuitry within said unit to determine whether a detected light level is above a specific threshold, and if so, the unit may be woken up, or activated to, for example, energize processing and communication circuitry by connection to the battery, or to a power source dependent on said battery. The optical wake-up circuit may consume extremely small amounts of battery power, facilitating a long shelf life for the monitoring unit.

A third embodiment will now be described, comprising a detailed, but exemplary, implementation of said optical wake-up circuit. The latter may comprise a first photodiode, a second photodiode, a capacitor, and a controlled switching element. Ambient, or other, light may fall onto the first photodiode, but may be prevented or blocked from reaching the second photodiode. For example, the second photodiode may be covered with a metal layer during manufacturing, thus reducing and/or preventing light from reaching it. The first photodiode, second photodiode and capacitor may be connected in such a way that current flowing through the first photodiode may divide proportionally to flow through said capacitor and said second photodiode. For example, current flowing from a positive battery terminal into a cathode of the first photodiode and out of its anode may divide and flow in proportional amounts, with one portion flowing into a cathode of the second photodiode and out of its anode towards a circuit ground, and the other portion flowing through said capacitor towards the ground. Further, the present invention teaches that the first photodiode may be chosen such that its characteristic dark-current value, that is the current flowing from cathode to anode when no or little light is incident on the photodiode, is less or substantially less than the characteristic dark-current value of the second photodiode.

Since said second photodiode is masked or covered to prevent, or to substantially prevent, light from reaching it, current flowing through it should always be at or near its characteristic dark-current value. In other words, only leakage current should flow through the second photodiode. According to the present invention, when the first photodiode is in a dark environment, for example, a product containing said first photodiode is still in packaging that blocks or substantially blocks light from reaching the first photodiode, all of the dark-current, or substantially all of the dark-current flowing through the first photodiode should flow through the second photodiode, given the characteristic dark-current values discussed above. Consequently, said capacitor may not charge up over time, with the capacitor voltage remaining at a low or zero value. The present invention teaches that said capacitor voltage may be used to close or open said switching element, or it may be used as input to a circuit controlling the switching element, or as an input to another associated circuit. For example, when the capacitor voltage is at a low or zero value, said switching element may be in an open state, or may be controlled to be in an open state, thereby preventing application of battery power, or of a voltage derived from a battery voltage, to an associated circuit or load connected to the wake-up circuit. In other words, when the first photodiode is in a dark environment, the capacitor may not charge up because all or almost all of the current flowing through said first photodiode should also flow through said second photodiode, and not through the capacitor, which may cause the switching element to remain in an open state, thereby disconnecting an associated load or circuit from the battery, or from a voltage derived from the battery voltage.

When a sufficient amount of light falls onto the first photodiode, for example when a product containing said first photodiode is removed from packaging that largely prevents light from reaching the first photodiode, current through the first photodiode may increase to a value larger than its characteristic dark-current value. According to the present invention, this may result in said capacitor being charged up, since the current flowing through the second photodiode should not increase beyond its characteristic dark-current value, with the remaining portion of the current through said first photodiode flowing through the capacitor. Once the capacitor voltage reaches a predetermined threshold, said switching element may close, or may be controlled to close, thereby connecting an associated load or circuit to the battery, or to a voltage derived from the battery voltage. In this exemplary manner, according to the present invention, a product containing the disclosed optical wake-up circuit may be woken up or activated. For example, a product containing said wake-up circuit may be removed from packaging and a user may shine a light, e.g., a light from a smartphone torch, onto the product. This may result in enough light falling onto said first photodiode to allow the described capacitor to fill up until its voltage crosses a predetermined threshold, upon which the switching element may be closed to cause the product to be energized with battery power, or with another power source dependent on said battery. The light used for wake-up may be coded in the one or other manner, according to the present invention, with a decoding or filtering circuit forming part of the optical wake-up circuit. This may be used to help prevent the optical wake-up circuit from erroneously waking up due to ambient light only, as an example.

The present invention teaches further that a circuit apart from said wake-up circuit, for example a circuit in said product containing a microcontroller, may also be used to control said switching element to cause it to open or close, thereby removing or applying battery power to selected circuits. Alternatively, the circuit which is apart from the wake-up circuit may control other elements of said wake-up circuit. For example, the wake-up circuit may have an input that can override the optical or other wake-up mechanism. This can be useful for example when the load says a microprocessor is woken up and must perform a series of tasks or function before the product can switch to low power again, or if it must in fact latch power on permanently. This can be described as an OR-function between the optical wakeup circuit and the processor output, i.e. if either one of the two is active then the power switch will stay closed. The same effect can also be achieved by combining the two outputs with a high signal (closing the switch) overriding a low signal. In addition, during manufacturing of said product it may be placed in a mode where the microcontroller remains active until said first photodiode has been in a dark environment, e.g., in the product packaging as described, for a sufficient period, whereafter microcontroller may cause said switching element to open, or cause another component to change states, thereby removing battery power, or a power source derived from said battery, from a number of circuits. For example, battery power may be removed from all circuits except the optical wake-up circuit after said sufficient period.

In another exemplary embodiment an optical wake-up circuit may function with current adjustment circuitry for the two (open and dark) photodiodes such as current mirrors. The current mirrors may also be used for subtracting the dark current from the open photodiode current to create a differential. The adjustment may allow for tuning the light level trip point for deciding to switch ON. Similarly, the trip level can also be adjusted through asymmetrical sizing of the photodiodes.

In another exemplary embodiment an optical wake-up circuit may function without said second photodiode, with current through said first photodiode flowing through the capacitor and wherein the latter may be prevented from charging up when the first photodiode is in a dark environment by a leakage current that equals, or substantially equals, the characteristic dark-current of the first photodiode. In other words, when the first photodiode is in a dark environment the dark-current through it is insufficient to charge said capacitor up, since a leakage-current similar to said dark-current causes continuous removal or dissipation of energy from the capacitor. For example, the leakage current may flow due to a resistor connected across the capacitor, or may be due to internal resistance of the capacitor and so forth.

The present invention teaches that said first and second photodiodes may have equal or similar dark-currents per unit area, also known as leakage-current per unit area, but the second photodiode may have a larger area and therefore a larger characteristic dark-current value. The present invention is not limited in this regard, and other means or methods may be used to ensure that the dark-current of said first photodiode is less, or substantially less, than that of said second photodiode. For example, current mirror structures may be used to ensure that dark-current of the first photodiode is less, or substantially less, than that of the second photodiode. This may be achieved in exemplary manner by using dark-current through said second photodiode as input current in a mirror structure, with an output current of the mirror structure determined by a mirror ratio, as is known in the art, and wherein said output current path may be used to sink the dark-current of said first photodiode.

Dark-currents or leakage-currents of photodiodes may exhibit dependence on temperature. Accordingly, the present invention teaches that a wake-up circuit as described may further employ temperature compensation to counteract said dependence. For example, a component or components with a temperature dependence inverse from that of the photodiodes may be used in the one or other manner to offset an increase or decrease in photodiode current due to temperature change. The present invention is not limited in this regard, and any relevant form of temperature compensation may be used.

In yet another exemplary, but more general embodiment of the present invention, a wake-up circuit for an electronic product may comprise a first circuit element, a second circuit element, a third circuit element, a fourth circuit element and a fifth circuit element. The fifth circuit element may comprise an output terminal connected to an associated circuit of said product, and may be used to place said associated circuit into a de-energized state, that is a sleep state, or into an energized state, that is a woken state. Said first to third circuit elements may each have at least a first and a second terminal, respectively. The first terminal of said first circuit element may be connected to a positive terminal of a battery in said product, with the second terminal of said first circuit element connected to both the first terminal of said second circuit element and the first terminal of said third circuit element. The second terminals of both the second and third circuit elements may be connected together and to a ground of the wake-up circuit, wherein said ground may be connected to a negative terminal of the battery. The junction of the second terminal of said first circuit element and the two first terminals of said second and third circuit elements may be connected to an input terminal of said fourth circuit element, with the fourth circuit element which may also be connected to said positive and negative terminals of the battery. The fourth circuit element may in turn have an output terminal which may be connected to an input of said fifth circuit element, the latter circuit element which may also be connected to said positive and negative terminals of the battery.

According to the present invention, the first circuit element may be sensitive to the one or other form of electromagnetic radiation, wherein the amount of radiation received may determine a value of current flowing through the first circuit element. This current may divide and flow proportionally through the second and third circuit elements towards said ground. Respective characteristics of the second and third circuit elements may be such that all or almost all of the current through said first circuit element flows through said third circuit element when said current is below a specific first value. Once current through the first circuit element rises above said first value, for example, due to an increase in radiation received by said first element, it may divide so that a larger portion flows through the second circuit element, while the current through said third circuit element may remain essentially constant, irrespective of any further increase in the current through the first circuit element. In other words, beyond a certain amount of radiation received by the first circuit element, more and more current may start to flow via said second circuit element, while a portion of current through the third circuit element stays the same.

Said second circuit element may be the one or other energy storage element, and may experience an increase in its voltage when current above a specific value flows through it. For example, the second circuit element may be a capacitor. Said fourth circuit element may control a voltage or current on its output terminal according to the voltage over said second circuit element, or according to current through it. The voltage or current on the output terminal of said fourth circuit element may in turn be used as input to the fifth circuit element, to control a voltage or current on the output terminal of the fifth circuit element. As described above, the output terminal of said fifth circuit element may be used to wake an associated circuit from a sleep mode.

In the preceding, as an example, the fourth circuit element may be the one or other circuit for monitoring a voltage or current of said second circuit element, and the fifth circuit element may be a switch, for example a MOSFET. In this case, the fifth circuit element may be only connected to the battery positive terminal and not to its negative terminal as well. Said associated circuit which is energized or de-energized by the fifth circuit element may be a microcontroller and/or communication circuit of said product. Further, the present invention teaches that the associated circuit may also be connected to said fourth or fifth circuit elements for controlling the states of these in the one or other manner. For example, when the associated circuit is a microcontroller circuit, it may control said fourth circuit element to keep the fifth circuit element in a state wherein the microcontroller circuit is energized.

Art practitioners may recognize the possibility in the preceding of omitting either the fourth or fifth circuit elements while maintaining similar wake-up functionality. For example, if said fourth circuit element is omitted, the voltage over the second circuit element may be directly applied as an input to the fifth circuit element to control wake-up of an associated circuit connected to the fifth circuit element. In this case, an output of said associated circuit may be directly connected to the junction of first terminals of said second and third circuit elements, for control of the voltage on said junction by the associated circuit when desired. Alternatively, said fifth circuit element may be omitted, with the output terminal of the fourth circuit element connected to said associated circuit, and, for example, applying battery power to it, or another voltage dependent on the battery voltage. Again, an output terminal of the associated circuit may be directly connected to said junction of first terminals of the second and third circuit elements, and used for control of the fourth circuit element by said associated circuit.

In another exemplary embodiment of the present invention, a wake-up circuit for a product may comprise first and second circuit elements characterized in that they do not draw current from a battery of said product, with second terminals of both said first and second circuit elements which may be connected together and to a circuit ground and a negative terminal of the battery. A first terminal of the first circuit element may be connected to a first terminal of the second circuit element, wherein the connection may be used to transfer energy from said first circuit element to said second circuit element. The latter may store energy thus received in the one or other electrical energy store until stored energy in said store crosses a first threshold. A third terminal of the second circuit element may be connected to a first input of a third circuit element of the wake-up circuit, and wherein said third terminal may be used to communicate said crossing of the first threshold to said third circuit element. The third circuit element may be connected between a positive terminal of said battery and the circuit ground, and may be used to connect an associated circuit in said product to the positive terminal via an output of the third circuit element, or to another voltage dependent on the battery voltage, thereby energizing or activating said associated circuit. For example, the associated circuit may be connected to the battery positive once said energy store crosses said first threshold, although the invention is not limited in this regard. A second input terminal of the third circuit element may be used by the associated circuit for control of the third circuit element, for example to ensure that the connection between said battery positive terminal and the associated circuit is maintained.

According to the present invention, said first circuit element in the directly preceding may be used to sense or receive the one or other form of electromagnetic radiation, for transfer to the energy store of said second circuit element. For example, the first circuit element may be a coil or inductor used as a secondary coil to receive magnetic energy from a coupled primary coil under control of a user for product wake-up or activation. Or the first circuit element may be the one or other radio frequency (RF) receiving element used to receive RF-energy emanating from a transmitter under control of a user for product wake-up. As another example, the first circuit element may be a small solar cell and used to receive ambient light or light directed by a user at said first circuit element for product wake-up. The present invention is not limited in this regard, and said first circuit element may be any element which can receive energy from another source under control of a user. A number of options for masking or shielding the first circuit element in the directly preceding may exist, according to the present invention. For example, if said first circuit element comprise a coil or inductive element, product packaging may comprise magnetic material to shield the first circuit element while packaged. As a more detailed example, if the first circuit element is a PCB coil, flexible, adhesive ferrite sheets may be used to cover the coil on one or both sides, thus substantially shielding it from magnetic fields, which may prevent unintentional product wake-up. As another example, if said first circuit element comprise the one or other RF-receiving element, conductive material in the product packaging may be used to detune it, or to shield it from RF radiation. Lastly, if said first circuit element comprise the one or other optical component for conversion of light to electrical energy, product packaging may mask it with material impermeable or substantially impermeable to light. The present invention is not limited in this regard, and said product packaging may use any material or structure to block or redirect electromagnetic energy from a sensor element used by wake-up circuits of the present invention.

Temperature measurements may also be used to determine when an electronic device should be woken up, according to the present invention. For example, a device embodying the invention may make use of temperature measurements with the one or other temperature sensor or probe such as a thermistor, thermopile and so forth, to determine when an ambient, or other, temperature changes as required or reaches a target range, which may result in device wake-up. In a more specific exemplary embodiment, a device may utilize a negative temperature coefficient (NTC) thermistor, as an example of a temperature probe, to measure ambient temperature. When a predetermined change in temperature within a predetermined period occurs, the device may transition from a low power or sleep mode to an active or high-power mode.

In a related exemplary embodiment, said device may further combine temperature measurements with capacitive sensing to determine when to wake up. In such an embodiment, the device may comprise, e.g., an NTC thermistor providing a temperature dependent voltage or current to a monitoring circuit of the device. The monitoring circuit may, but need not, selectively apply power to the NTC thermistor to take a temperature reading, wherein said selection may be used to reduce average power consumption of the device in a sleep or low power mode. Said monitoring circuit, or another circuit in communication with it, may additionally perform capacitive sensing to determine when a predetermined capacitance or change in capacitance is measured. Device wake-up may require the one or other temporal relationship between the measurement of said predetermined capacitance, or change in capacitance and measurement of a predetermined temperature, or change in temperature, by said monitoring device. To minimize power consumption of the device in said sleep or low-power mode, capacitive sensing measurements may be performed intermittently, for example at the same rate as the temperature measurements, or at a lower of higher rate.

A more specific exemplary embodiment where the directly preceding may be used to fashion a wake-up circuit in a disposable, adhesive blood glucose monitoring unit will now be described. In such a unit, temperature sensing may be used to detect when the unit is removed from packaging and placed against a user's skin, which should cause a measured temperature to change to the same range as a body temperature of a user. To ensure that the unit does not erroneously measure an ambient temperature in this range, for example when the packaged blood glucose unit is located within a hot environment, said unit may utilize capacitive sensing to confirm that the unit has been placed against the skin of the user. The present invention is not limited in this regard, and any relevant form of capacitive sensing may be used, for example self-capacitance sensing or mutual-capacitance sensing, with any number of required electrodes. The unit may also make use of differential capacitive sensing wear detection technology.

In yet another exemplary embodiment an electronic device may utilize two exposed contacts in a wake-up circuit, wherein selective immersion of said contacts in water or another liquid may cause a wake-up event. One of the contacts may be connected to a positive terminal of a battery of said device, while the other may be connected to the wake-up circuit, wherein the latter may be completely disconnected from said battery, barring any leakage currents, during a sleep mode. For example, the wake-up circuit may be disconnected from said battery via a semiconductor switch such as a MOSFET during sleep mode. When a product containing said device is packaged, the two contacts may be protected from liquids by the packaging. Once removed from the packaging, a user may immerse the two contacts, or the whole product, if relevant, into water or another liquid. This may allow sufficient conduction of current between the contacts to power said wake-up circuit after a period of immersion, after which the wake-up circuit may close said semiconductor switch to cause battery power to be applied with low losses to the wake-up circuit. Subsequently, the wake-up circuit may utilize an output terminal to cause said device to wake up and transition to an active or high-power state, whereupon the contacts or product may be removed from said water.

A two-contact based wake-up circuit as described may be advantageously applied to products such as disposable, adhesive blood glucose monitoring units, since these units are typically designed to be completely waterproof and to be worn while showering, swimming etc. In addition, the present invention teaches that a thin adhesive strip may be placed over said exposed contacts while packaged, to substantially prevent oxidation of the contacts.

In a related exemplary embodiment, the two exposed contacts described need not be immersed in water or another liquid after removal from product packaging. Instead, a user may touch the contacts for a specific period and/or with a specific pattern to cause product wake-up. Skin of a user thus placed on the contacts may serve as a galvanic connection between the contacts, similar to the water or liquid described before in the present disclosure. This may allow application of battery power, albeit through a fairly high resistance posed by the user's body part connected to said contacts, to a wake-up circuit. Similar to before, the wake-up circuit may then close an electronic switch after a period of touch, wherein said switch closure may cause application of battery power with low losses to said wake-up circuit. Subsequently, the wake-up circuit may utilize an output terminal to cause product wake-up.

To prevent inadvertent touch on said two contacts, or connection of material other than a human body part, causing an erroneous wake-up event, the present invention teaches that a user may be required to touch said contacts in the one or other pattern, for example perform a long touch to ensure sufficient application of battery power to the wake-up circuit, followed by a plurality of short touches to confirm intention. The present invention is not limited in this regard.

In one form of the invention there is provided an electronic device that must consume very low power when in a sleep mode compared to when it is operational, wherein said device comprises a battery, an optical wake-up circuit and additional circuitry, said wake-up circuit including a detector sensitive to light, and wherein said detector is connected to a switch circuit for selective connection of said battery to said additional circuitry of the device, said selection to connect the battery being dependent on an amount of light incident on said detector, being above a minimum threshold.

In a preferred application the device includes a blood glucose monitoring unit. The device may be configured so that packaging of the device, which during a shelf life period is in the sleep mode, substantially prevents light from reaching the detector. Such additional circuitry may latch power ON irrespective of the status of the wake-up circuit.

In one embodiment the detector includes a first and a second photodiode and current from either or both of said photodiodes is adjusted using electronic circuitry and said photodiodes are not active when the power is latched ON due to action from another source.

The light may be coded with pulses.

In another embodiment there is provided an optical wake-up circuit comprising a first photodiode, a second photodiode and a switching element, wherein said first photodiode receives light incident on the circuit and said second photodiode is masked to substantially prevent its reception of said light, and wherein the switching element is controlled according to a metric related to the differential between the current of the said first photodiode and the current of the second photodiode. The device may comprise a capacitor, wherein the current of said first photodiode flows into the capacitor and the current of the second photodiode flows out of said capacitor, and wherein the control of said switching element is based on the voltage of the capacitor going above a predetermined level.

The wake-up circuit may be used in a body parameter monitoring unit.

The wake-up circuit may be used in a product with packaging that substantially prevents light from reaching said first photodiode and wherein the current from one or both of the photodiodes is manipulated using current mirrors. The battery may be disconnected from another circuit while said packaging remains intact.

### BRIEF DESCRIPTION OF THE DRAWINGS

The invention is further described by way of examples with reference to the accompanying drawings in which:

FIG. 1 shows an exemplary first embodiment in the form of a circuit diagram.

FIG. 2 shows another exemplary embodiment in the form of an adhesive blood sugar monitoring unit with optical wake-up.

FIG. 3 shows another exemplary embodiment in the form of a detailed optical wake-up circuit.

FIG. 4 shows an exemplary embodiment in the form of a wake-up circuit.

FIG. 5 shows an alternative wake-up circuit embodiment.

FIG. 6 shows an exemplary thermistor and capacitive sensing based wake-up circuit embodiment.

FIG. 7 shows an exemplary wake-up circuit embodiment employing exposed contacts.

### DETAILED DESCRIPTION OF EMBODIMENTS

To further clarify the disclosure of the present invention, the following detailed description relating to the appended drawings is presented, but should not be construed as limiting to the claims of the invention as it is merely used to support clarity of disclosure. A large number of alternative embodiments may exist that fall within the spirit and scope of the present invention, as may be recognised by one skilled in the relevant arts.

Herein, “or” is used to convey inclusive and not exclusive, unless expressly indicated otherwise or indicated otherwise by context. Therefore, herein, “A or B” may mean “A, B, or both,” unless expressly indicated otherwise or indicated otherwise by context. In addition, “and” is used to convey both joint and several, unless expressly indicated otherwise or indicated otherwise by context. Therefore, “A and B” may mean “A and B, jointly or severally,” unless expressly indicated otherwise or indicated otherwise by context.

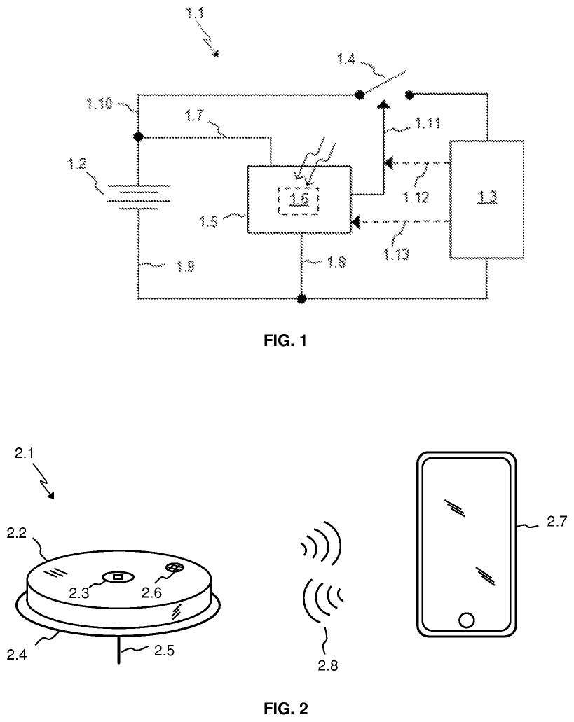

FIG. 1 shows an exemplary embodiment at 1.1, wherein an optical wake-up circuit 1.5 may be used to control application and/or removal of power from battery 1.2 to a load 1.3. The optical wake-up circuit 1.5 may be connected to a positive and negative terminal of battery 1.2 via respective connections 1.7 and 1.8, enabling circuit 1.5 to be continually or intermittently powered from battery 1.2, as required. An element 1.6 sensitive to ambient or other light, or to other forms of electromagnetic radiation, may be present in wake-up circuit 1.5. As illustrated, wake-up circuit 1.5 may have an output 1.11 which may be used to control a switching element 1.4, wherein the latter may be open or closed to respectively result in removal or application of power from battery 1.2 to load 1.3. The latter may be any electrical or electronic circuit. For example, load 1.3 may be a microcontroller circuit used to control the one or other product. Or it may be a power processing circuit such as a boosting or bucking DC-to-DC converter, as is known in the art. Switching element 1.4 may be used to connect or disconnect battery supply, or another power source, to or from said DC-to-DC converter, or it may be used to merely apply battery voltage, or another derived voltage, to a control pin or input terminal of said DC-to-DC converter. According to the present invention, when light, or other relevant electromagnetic radiation, is not incident on element 1.6 for a sufficiently long period, e.g., element 1.6 is in a dark environment for a sufficiently long period, wake-up circuit 1.5 may utilize output 1.11, or other means, not shown, to cause switching element 1.4 to be in an open state, resulting in the removal of battery power from load 1.3. For example, when element 1.6 is subjected to a dark environment for a sufficiently long period, a capacitor (not shown) within circuit 1.5 may discharge to a level which causes switching element 1.4, which may be a MOSFET, to transition to an open state. Therefore, switching element 1.4 may break the connection via 1.10 between a positive terminal of battery 1.2 and load 1.3. The connection 1.9 between a negative terminal of battery 1.2 and load 1.3 may remain in place.

Further, when element 1.6 is irradiated with relevant electromagnetic radiation for a sufficient period, for example, light of the correct wavelength and with sufficient strength falls onto element 1.6 for a sufficient period of time, wake-up circuit 1.5 may control switching element 1.4 via connection 1.11 to close, thereby applying battery power to load 1.3, or applying a voltage dependent on the battery voltage to load 1.3. For example, light falling onto element 1.6 may cause a capacitor (not shown) within circuit 1.5 to charge up to a predetermined level. When circuit 1.5 detects that the capacitor voltage is at said level, it may control switching element 1.4 to close, thereby applying power from battery 1.2 via connections 1.9 and 1.10 to load 1.3.

Naturally, the present invention is not limited to the preceding description and may also be embodied by a circuit where light incident on element 1.6 for a sufficiently long period may cause switching element 1.4 to open. In this case, an associated circuit or product may therefore be woken by removing light from sensing element 1.6 for a sufficient period, and may be entered into a sleep or low power state by applying light to said sensing element for a sufficient period.

As shown in FIG. 1, load 1.3 may also control the state of switching element 1.4 via connection 1.12. This may be apart from or in conjunction with the control exerted by wake-up circuit 1.5 via connection 1.11. For example, load 1.3 may be a microcontroller, or an energy processing circuit such as a boost DC-to-DC converter, and may keep switching element 1.4 in a closed state after manufacture and packaging until sensing element 1.6 has been in a dark environment for a predetermined period of time. The output from the unit 1.3 may also feed directly into the wake-up circuit 1.5 at a separate input with connection 1.13 and the unit 1.5 may contain the logic to create for example an “OR” function to enable switching on by the optical wake-up circuitry and then keeping the unit 1.3 powered by its own latching ON function, even if the light incidence onto the wakeup circuit is removed. This means a microcontroller or other circuit in 1.3 can also control the shut down, for example, after the product is being tested and packaged. It may also be an advantage to switch off the photodiode structure when the supply is latched ON from an external source, as it may reduce power consumption. This is especially true when the photodiodes are exposed to bright light as this can increase the current through the photodiode. The present invention is not limited in this regard, and load 1.3 may, for example, also be powered by a source other than battery 1.2 to selectively close switching element 1.4 to allow the one or other function to be performed. For example, inductive energy transfer may be used to selectively power load 1.3 while the product is in its packaging with sensor 1.6 blocked, with load 1.3 causing switch 1.4 to close, allowing remaining charge in battery 1.2 to be measured and communicated to another device. Thereafter load 1.3 may control switching element 1.4 to open again. This may, for example, be used to detect products with unacceptably depleted batteries and their removal from shelves in a shop. In an alternative embodiment, a product employing a circuit as illustrated in FIG. 1 may utilize two optical wake-up circuits similar to 1.5, with the first used to detect when the product in removed from its packaging, and the second to detect when a specific light source, for example a coded light source, is applied, causing the packaged product to measure and communicated its battery level to another device. Alternatively, a product may employ a single optical wake-up circuit similar to that illustrated by FIG. 1, but with two optical sensors, the first being similar to 1.6 and masked from ambient light when the product is packaged, and the second which may be sensitive to the one or other form of coded light only, and not masked when the product is packaged. A user may then illuminate said second sensor with the relevant coded light beam while the product is packaged, causing the product to wake up, determine its battery level-of-charge, communicate the same, and return to sleep.

FIG. 2 depicts another exemplary embodiment of the present invention at 2.1 in the form of a blood glucose or blood sugar monitoring device 2.2, with an adhesive patch 2.4 and a pin or needle 2.5. The monitoring unit 2.2 may comprise a receptacle 2.3 for receiving an applicator tool to insert needle 2.5 into the skin of a user, as is known in the art. A device such as a smartphone 2.7 may be used to communicate via, for example, an RF-link 2.8 with monitoring device 2.2. This may be used for configuration, control and to read blood sugar levels, amongst other things. Device 2.2 may comprise a window or aperture 2.6, which may be sealed to prevent ingress of liquids, gasses or other matter. Window 2.6 may be transparent, or substantially transparent to light, or to other forms of electromagnetic radiation as required, and may be used to facilitate use of an optical wake-up circuit as described by the present disclosure or similar to it. In other words, when blood glucose monitoring unit 2.2 is within its packaging, window 2.6 may be masked or covered, preventing light from reaching a wake-up circuit within said device in sufficient quantities to cause wake-up. This may allow monitoring unit 2.2 to remain in a low power or sleep state while in its packaging, which may extend shelf life. Once monitoring unit 2.2 is removed from its packaging, ambient or other light may illuminate a light sensor (not shown) within unit 2.2 to cause wake-up. Alternatively, a user may shine a light, for example a pulse-coded light beam emitted by a smartphone, onto window 2.6 once monitoring unit 2.2 is removed from its packaging, causing the unit to wake-up and enter a higher power consumption state.

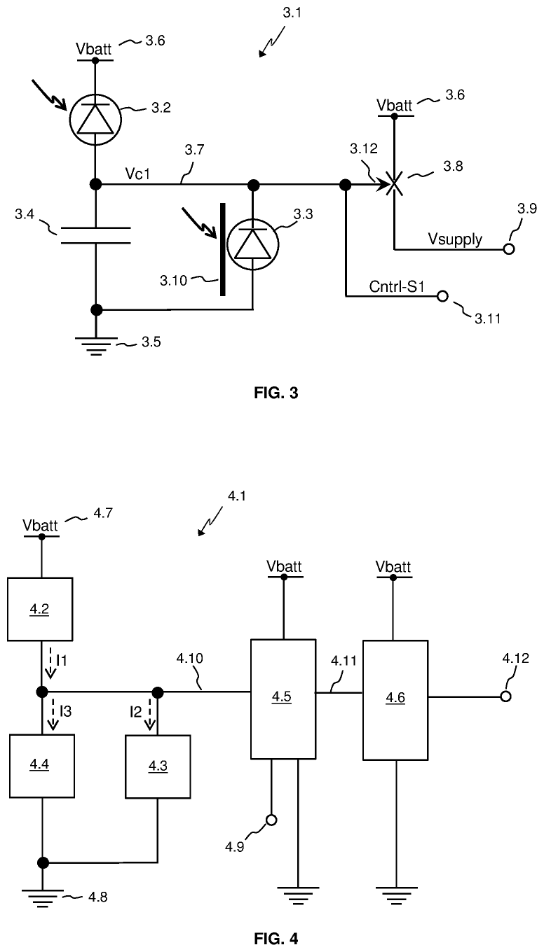

A detailed optical wake-up circuit embodiment is presented in exemplary manner in FIG. 3 at 3.1, comprising a first photodiode 3.2, a second photodiode 3.3, a capacitor 3.4 and a switching element 3.8. As shown, first photodiode 3.2 may be connected between a battery supply voltage 3.6 and capacitor 3.4, with second photodiode 3.3 connected in parallel to capacitor 3.4. A circuit ground 3.5 may be connected to a battery negative terminal (not shown), and to capacitor 3.4 and second photodiode 3.3 as shown. The latter may be masked with a member 3.10 to prevent or to substantially prevent light from reaching second photodiode 3.3. Therefore, any current flowing through 3.3 should be equal to or close to its characteristic dark-current or leakage-current.

First and second photodiodes 3.2 and 3.3 may be chosen such that the characteristic dark-current of the latter is more, or substantially more, than that of the former, according to present invention. Consequently, when no or little light is incident on the first photodiode 3.2, for example when a product utilizing the circuit at 3.1 is still within packaging that masks or blocks photodiode 3.2, all, or substantially all, of the current flowing from the battery at 3.6 via first photodiode 3.2 should also flow through via second photodiode 3.3 to ground 3.5, bypassing capacitor 3.4. This current may be equal to the dark- or leakage-current of first photo-diode 3.2, typically an extremely small value. As such, a battery connected to 3.6 should not deplete rapidly.

Due to said bypassing, capacitor 3.4 should not charge-up when photodiode 3.2 is within a dark environment for an extended period, and its voltage Vc1 on bus 3.7 may remain at a low or zero value. As is evident from FIG. 3, voltage Vc1 may be applied to switching element 3.8 at input terminal 3.12. When voltage Vc1 is below a first value, switching element 3.8 may be in a first state, and when voltage Vc1 is above said first value, switching element 3.8 may be in a second state, as is known in the art. As a non-limiting example, when Vc1 is at a low or zero value, switching element 3.8 may be in an open or non-conducting state, and when Vc1 is above said first value, switching element 3.8 may be in a closed, or conducting state, as is known in the art. Accordingly, the present invention teaches that when little or no light is incident on first photodiode 3.2 and capacitor 3.4 remains uncharged with Vc1 at a low or zero value due to dark-current of photodiode 3.2 flowing mainly or completely through photodiode 3.3, switching element 3.8 may remain in an open or non-conducting state. Consequently, terminal 3.9 is disconnected from the battery voltage Vbatt on bus 3.6, wherein terminal 3.9 may also be connected to a supply bus Vsupply of an associated circuit (not shown), for example to the supply bus of a microcontroller circuit (not shown) of a product comprising the optical wake-up circuit of FIG. 3. Alternatively, said associated circuit (not shown) may be the one or other energy processing circuit, for example a boost DC-to-DC converter, and terminal 3.9 may be connected to a voltage input pin of the converter (not shown), to a supply pin (not shown) or to an enable pin (not shown). When the voltage Vc1 on capacitor 3.4 is at a low or zero value, battery voltage Vbatt, or another voltage derived from it, may be disconnected from the exemplary pins of said converter.

Conversely, according to the present invention, when sufficient light falls onto photodiode 3.2, current through it from bus 3.6 towards ground 3.5 may increase to a first value which is substantially more than the characteristic dark-current of photodiode 3.3. Because the latter is masked from light by element 3.10, for example by an area of metal located over it, current through it from bus 3.7 towards ground 3.5 should be limited to its characteristic dark-current. As such, the first value of current through photodiode 3.2 may proportionally divide to flow through capacitor 3.4 and photodiode 3.3, which may cause capacitor 3.4 to charge up with a portion of the current through photodiode 3.2. When voltage Vc1 increases above a specific value due to said charging, switching element 3.8 may transition states due to application of voltage Vc1 at input terminal 3.12. For example, switching element 3.8 may transition from an open or non-conducting state to a closed or conducting state, thereby connecting terminal 3.9 to bus 3.6, with the supply bus Vsupply correspondingly connected to battery bus Vbatt. In other words, the present invention teaches that terminal 3.9 may be connected to bus 3.6 by shining a light of sufficient strength onto photodiode 3.2, and that the circuit of FIG. 3 may be used as an optical wake-up circuit in this exemplary manner. If said associated circuit (not shown) connected to terminal 3.9 comprise a DC-to-DC converter circuit, as an example, closure of switching element 3.8 as described in the directly preceding may result in application of battery voltage Vbatt, or another voltage derived from the battery voltage, to an input of said converter circuit, or to an enable pin of the converter circuit.

In addition, the present invention teaches that switching element 3.8 may be controlled via another terminal 3.11 with a signal Cntrl-S1 as well. Said control may be separate from or in conjunction with the control exerted by the circuit as described above. For example, terminal 3.11 may be used by a microcontroller (not shown) to control the state of switching element 3.8, wherein when Cntrl-S1 is above a specific value, switching element 3.8 may be in a closed or conducting state, and when Cntrl-S1 is below said specific value, switching element 3.8 may be in an open or non-conducting state.

It is to be appreciated that the present invention is not limited to the voltage Vc1 of capacitor 3.4 being applied directly to an input terminal of a switching element, for example to terminal 3.12 of 3.8, to effect control of the switching element based on the amount of light incident on photodiode 3.2. For example, a voltage or current, or another parameter, which is based on or derived from the voltage Vc1 may be applied to the one or other circuit (not shown), being digital or analog or a combination thereof, to control the switching state of a switching element such as 3.8, or to control an operational state of another component or circuit, based on the amount of light incident on photodiode 3.2.

The present invention teaches that an optical wake-up circuit as shown in FIG. 3, or reasonable alternatives thereof, may specifically be used in blood glucose monitoring units, in conjunction with the packaging of said units, to facilitate a sleep or low-power mode of the units when packaged, and a wake-up or transition to a higher-power or active mode of the units when removed from their packaging to allow light with specific characteristics to fall upon said wake-up circuit.

FIG. 4 depicts a more general exemplary embodiment of the present invention at 4.1, where a wake-up circuit may comprise a first circuit element 4.2, a second circuit element 4.3, a third circuit element 4.4, a fourth circuit element 4.5 and a fifth circuit element 4.6. The exemplary wake-up circuit may be connected to a positive terminal of a battery (not shown) via bus 4.7, supplying a voltage Vbatt. A ground 4.8 of the wake-up circuit may be connected to a negative terminal of the battery (not shown). As shown, first circuit element 4.2 may be connected to bus 4.7 on one side and to the parallel combination of second and third circuit elements 4.3 and 4.4 on the other, with said parallel combination connected to ground 4.8 at one end. Circuit element 4.2 may be sensitive to the one or other form of electromagnetic radiation coupling with it, with the value of a current I1 through element 4.2 which may be dependent on the amount of coupled radiation. As depicted, current I1 may divide into currents I2 and I3, to respectively flow through second circuit element 4.3 and third circuit element 4.4 towards ground 4.8, assuming negligible current flowing into fourth circuit element 4.5 via connection 4.10. Further, second circuit element 4.3 may be shielded from radiation coupling with first circuit element 4.2, or it may be insensitive to said radiation. When a product utilizing the wake-up circuit of FIG. 4 is packaged, the one or other element (not shown) of the packaging may mask first circuit element 4.2 from radiation and prevent said coupling. As a result, the value of 11 should only represent a leakage-current value. Due to the division into currents I2 and I3, this value of 11 may be too small to allow an energy storage element (not shown) within third circuit element 4.4 to store sufficient energy to cause a significant increase in the voltage of bus 4.10. Consequently, fourth circuit element 4.5 may control fifth circuit 4.6 via interconnection 4.11 to not connect terminal 4.12 to battery voltage Vbatt, or to another voltage derived from Vbatt, based on the low value of the voltage on bus 4.10. Terminal 4.12 may be used to supply power or a relevant signal to an associated circuit (not shown) of said product, similar to that described before. The associated circuit (not shown) may be, as examples, a microcontroller circuit, a communication circuit or an energy processing circuit such as a boosting or bucking DC-to-DC converter circuit.

When sufficient amounts of radiation couple with first circuit element 4.2, current I3 may become large enough, notwithstanding the value of current I2, to allow said storage element (not shown) in third circuit element 4.4 to store sufficient energy for bus 4.10 to exceed a first threshold voltage. According to the present invention, current I2 may be limited by the characteristics of second circuit element 4.3, or through another mechanism, to a specific value, notwithstanding the voltage upon bus 4.10, at least up to a breakdown voltage value, whereafter current I2 may increase rapidly with increasing voltage over element 4.3. Consequently to said first threshold being exceeded, fourth circuit element 4.5 may control fifth circuit element 4.6 via interconnection 4.11 to connect terminal 4.12 to battery voltage Vbatt, or to another voltage derived from Vbatt, thereby energizing or activating an associated circuit connected to terminal 4.12. Interconnection 4.11 between fourth circuit element 4.5 and fifth circuit element 4.6 need not be a galvanic or wired connection, but may also be the one or other wireless connection, if so required.

Fourth circuit element 4.5 may have a second input terminal 4.9, which may be used by said associated circuit (not shown) to control the fifth circuit element 4.6 via said fourth circuit element 4.5, as exemplary embodiment. For example, said associated circuit (not shown) may be a microcontroller or DC-to-DC converter circuit that utilizes terminal 4.9 to ensure that fifth circuit element is maintained in a state where terminal 4.12, connected to a supply of the microcontroller or the one or other pin of said DC-to-DC converter, is connected to battery voltage Vbatt or to a voltage derived from it.

In an alternative exemplary embodiment, fourth circuit element 4.5 may monitor current I3 into third circuit element 4.4 instead of the voltage on bus 4.10 to determine when to control fifth element 4.6 to cause wake-up of a product employing the wake-up circuit disclosed by FIG. 4.

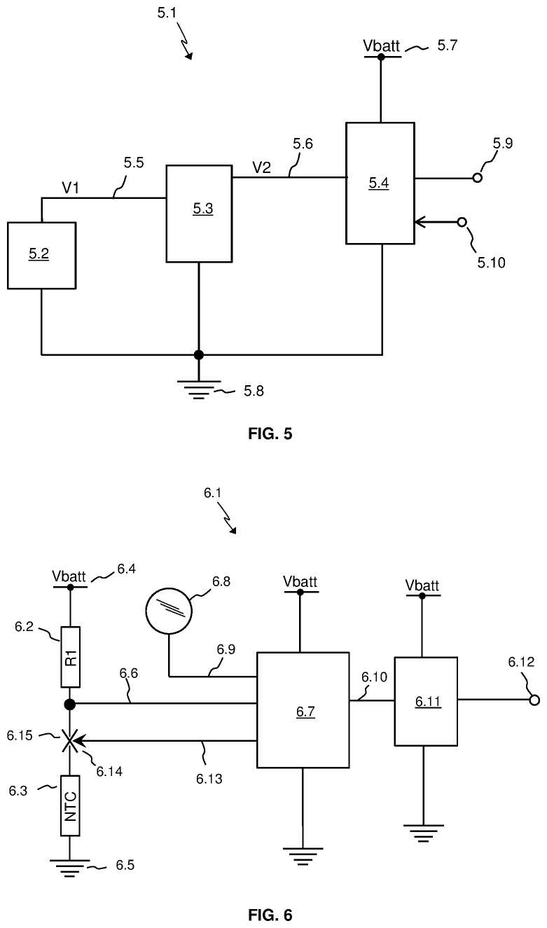

Yet another exemplary embodiment in the form of a wake-up circuit for an electronic product is presented at 5.1 in FIG. 5, comprising a first circuit element 5.2, a second circuit element 5.3 and a third circuit element 5.4, interconnected as shown. Said first circuit element 5.2 may be a component sensitive to electromagnetic radiation such that a voltage V1 on a connection 5.5 may increase or decrease dependent on an amount of radiation coupled to circuit element 5.2. As a first more specific example, first circuit element 5.2 may be an inductor or coil coupled to a second coil, the latter which may be under control of a user. A magnetic field emanating from said second coil may couple with circuit element 5.2 for transfer of magnetic energy. As a second specific example, first circuit element 5.2 may be a light component such as a small solar cell for reception of ambient or other light. As a third specific example, first circuit element 5.2 may be a component for reception of RF-energy from an RF-transmitter under control of a user. In each of these examples, voltage V1 may increase or decrease dependent on the amount of energy coupled to first circuit element 5.2. For example, if a product employing the wake-up circuit of FIG. 5 is located in packaging which masks or shields first circuit element 5.2 from relevant electromagnetic radiation, be it a magnetic field, visible light or RF-frequency signals, voltage V1 may be at a low value. Conversely, when said product is removed from the packaging first circuit element 5.2 may receive sufficient amounts of electromagnetic radiation that voltage V1 may increase substantially, wherein said increase may be used to facilitate wake-up of said product.

Second circuit element 5.3 may receive voltage V1 at input 5.5, and may comprise the one or other electrical energy store for storage of electrical energy received from first circuit element 5.2. Second circuit element 5.3 may further, but need not, comprise circuitry to generate a second voltage V2 on output 5.6, wherein said second voltage may be higher than V1. As depicted, voltage V2 may be applied to third circuit element 5.4 via connection 5.6, for control of the state of third circuit element 5.4. For example, when voltage V2 is below a first threshold value, third circuit element 5.4 may be in a state where battery voltage Vbatt at 5.7, or another voltage derived from the battery voltage, is not connected to output terminal 5.9. Conversely, when voltage V2 increases beyond said first threshold due to electromagnetic radiation coupled to first circuit element 5.2, third circuit element 5.4 may consequently transition to a state where terminal 5.9 is connected to battery voltage Vbatt, or to another voltage derived from it, for example through closure of a switching element (not shown) in third circuit element 5.4. Terminal 5.9 may be connected to a supply terminal of an associated circuit (not shown), with the latter also connected to wake-up circuit ground 5.8. In this exemplary manner, the associated circuit may be woken up, or energized, when the first circuit element 5.2 receives sufficient electromagnetic radiation for voltage V2 to increase above said first threshold. For example, the associated circuit (not shown) may be a DC-to-DC converter circuit, and terminal 5.9 may be used to enable or disable operation of the converter.

Further, similar to before, third circuit element 5.4 may have an input terminal 5.10 which may be used by said associated circuit (not shown) to control the state of the third circuit element 5.4, either separate from or in conjunction with the control exerted by second circuit element 5.3 via voltage V2.

FIG. 6 depicts an exemplary embodiment in the form of a temperature dependent wake-up circuit at 6.1, wherein an NTC thermistor 6.3 may be connected in series with a resistor 6.2 between a battery voltage 6.4, or another voltage derived from said battery voltage, and a circuit ground 6.5. A monitoring circuit 6.7 may monitor the voltage over NTC thermistor 6.3 via connection 6.6, and may further control the one or other switching element 6.15 via an input terminal 6.14 of said element through a voltage or current on or in a connection 6.13. This may be done, for example, to minimize power consumption by resistor 6.2 and NTC thermistor 6.3 through intermittent closure of switching element 6.15. It should be noted that the present invention may be practised without the use of switching element 6.15, in which case resistor 6.2 and NTC thermistor 6.3 may be continuously connected to each other. In addition, NTC thermistor is merely shown as an example of a temperature sensor, and any relevant alternative may be used in its place.

During a sleep-mode of the wake-up circuit depicted in FIG. 6, monitoring unit 6.7 may be in an extremely low power mode, wherein it may intermittently check for a predetermined temperature, or a predetermined change in temperature, measured with NTC thermistor 6.3. Monitoring circuit 6.7 may control circuit element 6.11 via connection 6.10 such that circuit element 6.11 may cause an associated circuit (not shown) connected to terminal 6.12 to be deactivated or in a low-power sleep mode when said predetermined temperature, or change in temperature, is not observed. Although circuit element 6.11 is shown as connected to both a battery voltage Vbatt and to circuit ground, art practitioners may appreciate that this need not be so. For example, if circuit element 6.11 is switch such as a MOSFET, the connection to ground is not required, and circuit element may be used to either connect or disconnect terminal 6.12 to the battery voltage Vbatt, or to a voltage derived from it, thereby activating or deactivating an associated circuit (not shown) connected to terminal 6.12. The present invention is not limited in this regard. For example, if said associated circuit (not shown) is a DC-to-DC boost converter circuit, terminal 6.12 may be connected to a voltage input pin (not shown) or an enable pin (not shown), to facilitate activation or deactivation of said converter via terminal 6.12.

When said predetermined temperature, or change in temperature, is measured via NTC thermistor 6.3, monitoring circuit 6.7 may use this information, optionally along with other parameters, to change states causing a predetermined change in the voltage on, or current through, interconnection 6.10 with circuit element 6.11. This may cause a change in the voltage on, or current through, terminal 6.12, which may be used to wake an associated circuit (not shown) connected to said terminal. In this manner the circuit in FIG. 6 may be used to wake a product comprising said circuit when the predetermined temperature, or change in temperature, is measured.

The exemplary embodiment of FIG. 6 may be advantageously applied to a product such as a blood glucose monitoring unit, similar to that shown in FIG. 2, wherein the unit is in a deep sleep, drawing very small amounts of battery current, until it is placed on a user's skin, whereafter the measured temperature, or change in temperature, caused by the user's body temperature may result in the unit waking up.

To further improve functionality robustness, the present invention teaches that a wake-up circuit such as depicted in FIG. 6 may use capacitive sensing in conjunction with the described temperature monitoring. To this end, a capacitive sensing electrode 6.8 may be connected to monitoring circuit 6.7 via connection 6.9. For example, electrode 6.8 may be used for self-capacitance measurements by circuit 6.7, although the invention is certainly not limited to this, with mutual-capacitance measurements a viable, but exemplary, alternative. According to the present invention, monitoring circuit 6.7 may also perform capacitance measurements intermittently, for example to save battery power while in a sleep mode. The intermittency of said measurements may be based on any relevant temporal schedule, for example the capacitance measurements may be performed together with the described temperature measurements, in whichever manner required. The present invention teaches that monitoring circuit 6.7 may require measurement of both a predetermined temperature, or a change in temperature, and a predetermined capacitance, or a change in capacitance, within a specific period, or other temporal relationship, before an associated circuit (not shown) connected to terminal 6.12 may be woken up, activated or energized with battery power, whichever the case may be.

It is to be appreciated that the depiction of FIG. 6 is purely exemplary, and should not be used to unduly limit the invention. For example, monitoring circuit 6.7 need not perform said capacitance measurements, and another circuit (not shown) may perform said measurements and only communicate the results thereof, or capacitance values or other related parameters, to circuit 6.7 via any wired or wireless connection, as may be required.

Reverting to the previously mentioned blood glucose monitoring unit use of the circuit depicted in FIG. 6. When said user applies the unit to his/her skin, the predetermined temperature, or change in temperature, may be measured. But it is foreseeable that such a temperature or change in temperature may also be caused by a hot environment in which a packaged blood glucose monitoring unit is located. However, if capacitive sensing as described and depicted in FIG. 6 is utilized to confirm placement of the unit on a user's skin when said predetermined temperature, or change in temperature, is measured, the chance of erroneous operation may be severely minimized.

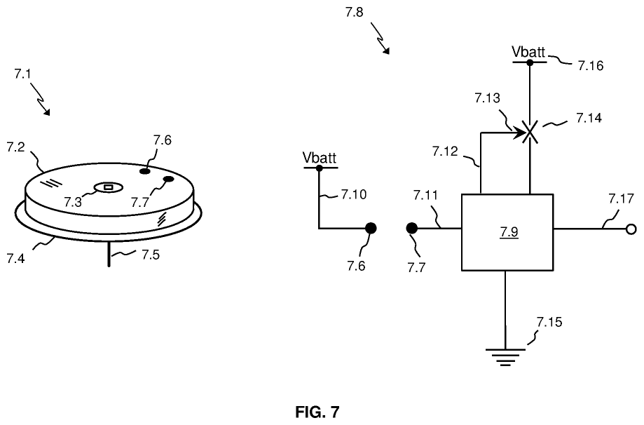

Yet another exemplary embodiment of a blood glucose monitoring unit with a potentially low-cost wake-up circuit is presented in FIG. 7. The blood glucose monitoring unit is merely used as an example product, and the present invention should not be limited to this, with a large number of alternative products which may potentially use a circuit as shown in FIG. 7 and described hereafter. An exemplary blood glucose monitoring unit is shown at 7.1 in FIG. 7, similar to that shown in FIG. 2, with a unit body 7.2, an adhesive patch 7.4, a pin or needle 7.5 and a receptable for an applicator tool 7.3. In addition, the unit at 7.1 may comprise two exposed contacts 7.6 and 7.7, which may be used to wake the unit from a sleep mode when it is removed from its packaging. An exemplary wake-up circuit that may be used by a unit as shown at 7.1 is shown at 7.8, with exposed contacts 7.6 and 7.7 respectively connected to a battery voltage Vbatt and to an input terminal 7.11 of a monitoring circuit 7.9. The latter may further be connected to a circuit ground 7.15 and to a battery voltage Vbatt at 7.16 via a controlled switch 7.14. As shown, monitoring circuit 7.9 may control switch 7.14 via output 7.12 which may be connected to switch input terminal 7.13. For example, switch 7.14 may be a MOSFET and terminal 7.13 may be a gate thereof, as is known in the art. When switch 7.14 is in an open state, monitoring circuit 7.9 may be disconnected from battery voltage Vbatt at 7.16, without any current drawn by circuit 7.9, barring leakage currents. To cause a wake-up event, according to the invention, a user may touch or press onto exposed contacts 7.6 and 7.7. Due to skin resistance, this may effectively connect contacts 7.6 and 7.7 together via said skin resistance, the latter typically being on the order of a few hundred Ohms to kilo-Ohms, although the invention should not be limited to this range. Accordingly, monitoring circuit 7.9 may be connected to battery voltage Vbatt via said skin resistance, which may allow a small current to flow into circuit 7.9. This current may be used in the one or other manner to control switch 7.14 via connection 7.12 and terminal 7.13 to close, thereby connecting monitoring circuit 7.9 to battery voltage Vbatt with minimal losses. Subsequently, monitoring circuit 7.9 may use its output terminal 7.17 to wake an associated circuit (not shown) connected to said terminal up. For example, terminal 7.17 may be connected to a microcontroller input pin (not shown), or to an enable pin (not shown) or voltage input pin (not shown) of a DC-to-DC converter (not shown)

Monitoring circuit 7.9 may utilize a first capacitor (not shown) and first resistor (not shown) connected in parallel with each other, and between terminal 7.11 and circuit ground 7.15 to effect control of switching element 7.14 when a user touches contacts 7.6 and 7.7 for a sufficient period. The value of said first resistor may be chosen such that it is smaller or significantly smaller than the skin resistance between contacts 7.6 and 7.7 during said touch. Consequently, if only a light touch, or other inadvertent connection, is present between contacts 7.6 and 7.7, most or all of the current flowing via said contacts should also flow via said first resistor to ground, bypassing the first capacitor in parallel with said first resistor. The amount of charge stored in the first capacitor may therefore remain at a low or zero value, with a low or zero voltage over said capacitor. This may prevent monitoring circuit 7.9 from closing switch 7.14 via connection 7.12, and thereby prevent activation of wake-up of an associated circuit (not shown) connected to terminal 7.17. Conversely, when a user press down with sufficient force onto contacts 7.6 and 7.7, more current may flow from the battery due to a lower resistance between said contacts, causing the first capacitor to fill up, wherein its consequent increase in voltage may be used by circuit 7.9 in the one or other manner to close switch 7.14, allowing activation of said associated circuit.

The present invention further teaches that exposed contacts other than those depicted at 7.1 may be used in the embodiment described above. For example, contact 7.6 may be located on a back of a blood glucose monitoring unit, or on its side, and contact 7.7 may form part of the pin or needle inserted into a user's skin. What is paramount is that two exposed contacts may be used to facilitate a connection via a body or body part of a user, and wherein said connection may then be used to cause activation of the unit, or of another product.

The present invention is not limited to use of skin resistance and user touch events as described in the directly preceding. For example, an embodiment as depicted in FIG. 7 may be immersed in water, or another liquid with sufficient conductivity, to cause a wake-up event. In this case, contacts 7.6 and 7.7 may be immersed in water for a period, wherein the water may provide a galvanic connection between the two contacts. This may allow battery voltage Vbatt to be connected to monitoring circuit 7.9 via the water. As a result, a small current may flow between contact 7.6 and 7.7, which may allow sufficient energy to be transferred from said battery to circuit 7.9 for closure of switch 7.14, with subsequent activation of an associated circuit (not shown) connected to terminal 7.17, similar to that described before. Since blood glucose monitoring units are typically designed to be waterproof for use while swimming or showering, an embodiment as described in the directly preceding may be advantageous.

1. An electronic device that must consume very low power when in a sleep mode compared to when it is operational, wherein said device comprises a battery, an optical wake-up circuit and additional circuitry, said wake-up circuit including a detector sensitive to light, and wherein said detector is connected to a switch circuit for selective connection of said battery to said additional circuitry of the device, said selection to connect the battery being dependent on an amount of light incident on said detector, being above a minimum threshold.

2. The device of claim 1 being a blood glucose monitoring unit

3. The device of claim 1, wherein packaging of the device, which during a shelf life period is in sleep mode, substantially prevents light from reaching the detector.

4. The device of claim 3, wherein the battery is disconnected from said additional circuitry while said packaging remains intact.

5. The device of claim 4, wherein the additional circuitry is configured to latch power ON irrespective of the status of the wake-up circuit.

6. The device of claim 5, wherein the detector includes a first and a second photodiode and wherein current from either or both of said photodiodes is adjusted using electronic circuitry and wherein said photodiodes are not active when the power is latched ON due to action from another source.

7. The device of claim 1, wherein the light is coded with pulses.

8. An optical wake-up circuit comprising a first photodiode, a second photodiode and a switching element, wherein said first photodiode receives light incident on the circuit and said second photodiode is masked to substantially prevent its reception of said light, and wherein the switching element is controlled according to a metric related to the differential between the current of the said first photodiode and the current of the second photodiode.

9. The wake-up circuit of claim 8, comprising a capacitor, wherein the current of said first photodiode flows into the capacitor and the current of the second photodiode flows out of said capacitor, and wherein the control of said switching element is based on the voltage of the capacitor going above a predetermined level.

10. The wake-up circuit of claim 8, wherein the switching element is used to selectively connect a battery to another circuit, dependent on said amount of light falling onto the photodiodes, or on an signal from said another circuit.

11. The wake-up circuit of claim 8 used in a body parameter monitoring unit.

12. The wake-up circuit of claim 11, wherein said unit comprises a blood glucose monitoring unit.

13. The wake-up circuit of claim 10 used in a product with packaging that substantially prevents light from reaching said first photodiode and wherein the current from one or both of the photodiodes is manipulated using current mirrors.

14. The wake-up circuit of claim 13, wherein the battery is disconnected from said another circuit while said packaging remains intact.

15. A wake-up circuit which includes a battery, a switch, an additional circuit and a detector which, responsive to incident light above a threshold level, closes the switch thereby to connect the battery to the additional circuit.
